(19) United States
(12) Patent Application Publication
(10) Pub. No.: US 2008/0087549 A1
(43) Pub. Date: Apr. 17, 2008

(54) Additive For Copper Plating And Process For Producing Electronic Circiut Substrate Therewith

(75) Inventors: Hiroshi Ishizuka, Kanagawa (JP); Nobuo Sakagawa, Kanagawa (JP); Ryoichi Kimizuka, Kanagawa (JP); Wei-ping Dow, Yunlin (JP)

Correspondence Address: OBLON, SPIVAK, MCCLELLAND MAIER & NEUSTADT, P.C., 1940 DUKE STREET, ALEXANDRIA, VA, 22314, US

(73) Assignee: EBARA-UDYLITE CO.,LTD., Tokyo (JP)

(21) Appl. No.: 11/573,718

(22) PCT Filed: Aug. 18, 2004
(86) PCT No.: PCT/JP04/11846

§ 371 Date: Jul. 30, 2007

(51) Int. Cl. C25D 5/02; C07D 257/04; C07D 245/02

(52) U.S. Cl. 205/125; 106/1.26; 524/89; 548/250; 548/254; 534/885

(57) ABSTRACT

An additive for copper plating comprising, as an effective ingredient, a nitrogen-containing biphenyl derivative represented by the following formula (I):

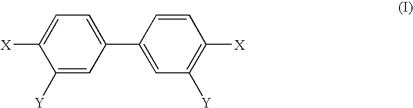

[wherein X represents a group selected from the following groups (II)-(VII):

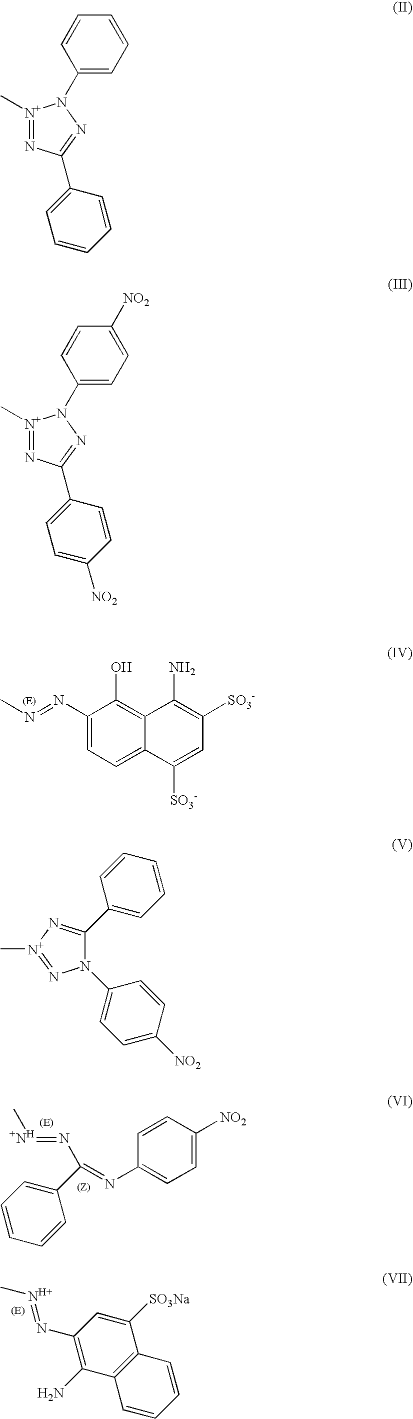

and Y represents a lower alkyl group, lower alkoxy group, nitro group, amino group, sulfonyl group, cyano group, carbonyl group, 1-pyridyl group, or the formula (VIII):

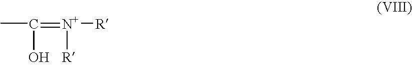

(wherein R′ represents a lower alkyl group)], a copper plating solution formed by adding the additive for copper plating to a copper plating solution containing a copper ion ingredient and an anion ingredient, and a method of manufacturing on an electronic circuit substrate having a fine copper wiring circuit, which comprises electroplating in the copper plating solution using as the cathode an electronic circuit substrate in which fine microholes or microgrooves in the shape of an electronic circuit are formed on the surface.

The additive for copper plating can fill through holes or via holes at a micron or sub-micron level even in a case where it consists of one component, and the copper plating solution using the additive for copper plating can be prepared and handled extremely easily and can stably fill the through holes or via holes for a long time.

### TECHNICAL FIELD

The present invention concerns an additive for copper plating, a copper plating solution containing the same, and a method of manufacturing an electronic circuit substrate using the copper plating solution and, more specifically, it relates to an additive for copper plating capable of filling through holes or blind via holes even upon use of one type of such additive, a copper plating solution containing the same, and a method of manufacturing an electronic circuit substrate such as a semiconductor substrate or a printed circuit substrate (PCB) using the copper plating solution.

### BACKGROUND ART

Keeping in step with size reductions and diversifications of electronic parts, there has also been demand for decreasing the thickness and decreasing the size of existent semiconductor wafers or IC circuit constituent substrate. Particularly, as Ball Grid Array (BGA) and Chips Scale Packaging (CSP) have come into general use, the size of the IC substrate has decreased rapidly and a number of electronic parts can be mounted on a small area.

In existent manufacturing techniques for IC copper wiring plate and PCB with high density wiring, a method of completely filling with copper plating has become predominant for inter-layer connection using vias or trenches, because this enhances the connection reliability and sealing property. However, since the vias or trenches are designed on the micron or sub-micron scale, there has been demand for a technique capable of preventing occurrence of filling failure, voids or seam voids.

Usually, two kinds of methods have been used for such via (through hole) filling electric copper plating. One of them is a method of bottom up filling with pulse or reverse pulse potential. Another is a method of using a DC current, but in this case multiple additives have to be added together into the plating solution.

Additives added in the plating solution are generally classified into three types: a suppressor, a leveler and a brightener.

Among them, a nonionic high molecular polymer is mainly used as the suppressor ingredient. The ingredient suppresses copper plating and has an effect of remarkably suppressing plating deposition on the surface of a material to be plated. Further, a nitrogen-containing compound (N⁺functional group) is used mainly as the leveler ingredient and it also suppresses plating. Since the ingredient contains a functional group as a cation, it tends to suffer from the effect of a current distribution. That is, since the ingredient is adsorbed preferentially to a region of high current distribution, it has an effect of suppressing occurrence of voids. Since the leveler ingredient has a strong diffusion controlling property and is adsorbed greatly on the plating surface of a thin diffusion layer so as to suppress deposition of plating, thereby preferentially growing plating in via holes or in through holes with a relatively thick diffusion layer, via-filling or through hole-filling is possible. Further, a sulfur-containing compound is mainly used as the brightener ingredient; it is bonded with copper ions in the via thereby providing an effect of relatively promoting deposition of plating in the via than that at the surface suppressed by the suppressor. With the synergistic effects of the additives, plating in the via, which is essentially a low current portion and where plating material hardly deposits, can be promoted.

While studies for filling have been progressed based on the combination of properties of each of such ingredients described above, a multi-component additive involves a problem that analysis of them is difficult and the quality control is difficult in view of practical use. Further, in a case where multi-component additives are present, the concentration of organic materials taken into the plated copper film also increases, which sometimes causes degradation of the film physical property. For the reasons described above, there has been demand for making the additive component simpler.

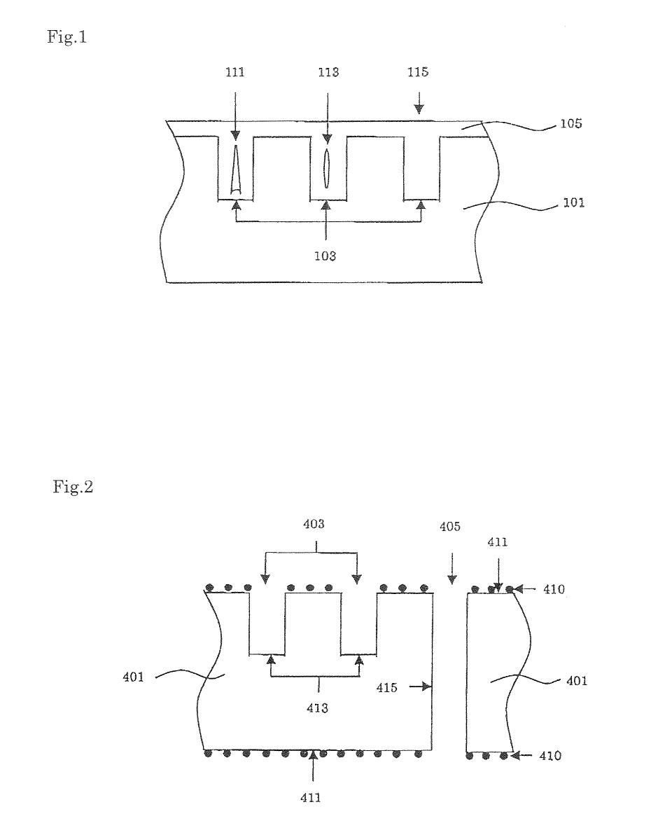

By the way, a cross sectional view of an IC substrate with via-hole filling copper plating as fabricated by an existent technology is schematically shown in FIG. 1. When an attempt is made to completely fill a blind via hole (103) of an IC substrate (101) by copper plating, it is known that three types of results are observed in the metal (copper) layer (105): a void (111), seams (113), and super filling (115).

It is difficult to completely eliminate the voids or seams, for example, only by current control such as pulse or reverse pulse. However, it has been known since 1966 that the combination of certain additives promotes growing of plating more at the bottom than at the surface of the hole, thereby reducing the voids or seams (Patent Documents 1 and 2). In the methods, bottom up deposition has been attained by using mercaptan compounds, PEG, chlorine ions and polycyclic compounds (Janus Green B; JGB) as the additives.

The mercaptan compound is used usually as the brightener in the filling plating. Specifically, bis-(3-sulfopropyl)disulfide disodium; SPS or 3-mercapto-1-propane sulfonate; MPS is used mainly. SPS and MPS are changed reversibly to each other during plating. An —SH group of the MPS bonds with copper ions to form a compound, thereby promoting the reduction reaction of the copper ions and improving the deposition rate of copper. Further, SPS and MPS are intensely adsorbed on the surface of the electrode during plating. At the electrode surface, MPS is formed by reduction of SPS. Since the reduction reaction from Cu²⁺to Cu caused by MPS and the reaction where the oxidized MPS returns to SPS occur simultaneously and repetitively, the rate of monovalent copper formation is improved. That is, copper deposition rate is improved.

Further, while a filling process using phthalocyanine compound (Alcian Blue) presented by Laudau U., et al. (Patent Documents 3 and 4) can be used for filling plating in semiconductors, it is not yet applied to PCB.

At present, many additives for via filling have been developed and used generally, but filling by copper plating is difficult for through holes of PCB or IC substrates and, in the existent method, a method of filling with a conductive paste or resin after copper plating has been adopted.

However, since the methods sometimes cause voids or peeling from hole inner walls due to the limited electroconductivity or volumic change after filling, etc., the reliability will be improved outstandingly if the inside of the through holes is filled with electroplating as is done in the via holes. For the reasons described above, there has been demand for the development of an additive for copper sulfate plating with simple composition, which is capable of completely filling via and through holes by a simple additive composition.

[Patent Document 1] U.S. Pat. No. 3,267,010

[Patent Document 2] U.S. Pat. No. 3,288,690

[Patent Document 3] U.S. Pat. No. 6,610,191

[Patent Document 4] U.S. Pat. No. 6,113,771

### DISCLOSURE OF THE INVENTION

### Problems that the Invention is to Solve

Accordingly, there has been demand for developing a technique capable of completely filling blind via holes, through holes, etc. at a micron or sub-micron level in semiconductor substrates or PCBs by copper plating using a simple additive and it is a subject of the present invention to provide such a technique.

### Means for Solving the Problems

The inventors have made an earnest study on a copper plating solution capable of filling microholes or micro grooves such as blind via holes or through holes while using as few additive as possible and, as a result, have found that the blind via holes, through holes, etc. described above can be filled fully by the addition of a specified nitrogen-containing biphenyl derivative to the copper plating solution, to accomplish the invention.

Specifically, the invention provides an additive for copper plating comprising, as an effective ingredient, a nitrogen-containing biphenyl derivative represented by the following formula (I):

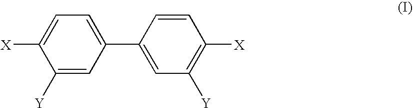

[wherein X represents a group selected from the following groups (II)-(VII):

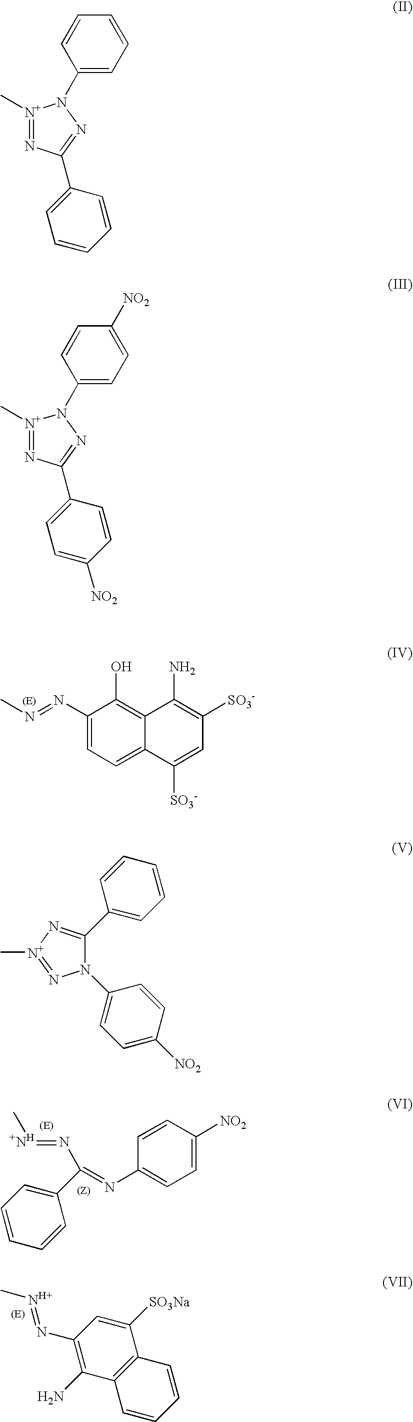

and Y represents a lower alkyl group, lower alkoxy group, nitro group, amino group, sulphonyl group, cyano group, carbonyl group, 1-pyridyl group, or the formula (VIII):

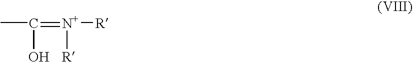

(wherein R′ represents a lower alkyl group)].

Further, the invention provides a copper plating solution formed by adding an additive for copper plating comprising, as an effective ingredient, a nitrogen-containing biphenyl derivative represented by the formula (I) to a basic composition of copper plating solution containing a copper ion ingredient and an anion ingredient.

Further, the invention provides a method of manufacturing an electronic circuit substrate having a fine copper wiring circuit, which comprises electroplating in a copper plating solution using, as a cathode, an electronic circuit substrate in which fine microholes or microgrooves in the shape of an electronic circuit wiring are formed on the surface.

### Effect of the Invention

The additive for copper plating according to the invention can fill through holes or via holes at a micron or sub-micron level even in a case where it comprises one component Accordingly, the copper plating solution using this additive for copper plating can be prepared and handled extremely easily and can stably fill the through holes or via holes for a long time.

### Embodiment of the Invention

The additive for copper plating of the invention comprises, as an effective ingredient, the nitrogen-containing biphenyl derivative represented by the formula (I) described above.

In the formula (I), the lower alkyl group or alkoxy group for Y preferably includes 1 to 3 carbon atoms, and may be branched. Further, the sulfonyl group or the carboxyl group may be free groups or groups forming salts with alkali metals such as sodium.

The nitrogen-containing biphenyl derivatives (I) are known compounds, or can be produced easily in accordance with known methods for preparing compounds.

For example, the nitrogen-containing biphenyl derivative (I) can be produced generally in accordance with the following formula (X):

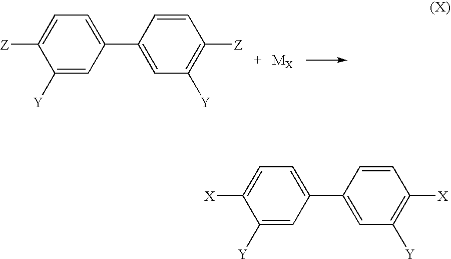

(wherein X and Y each has the meanings described above, M represents a hydrogen atom or an alkali metal or alkaline earth metal atom such as sodium, lithium and magnesium, and Z represents a halogen atom).

Among the nitrogen-containing biphenyl derivatives (I) , that in which the group X is the formula (II) and the group Y is H, that in which the group X is the formula (III) and the group Y is —OCH₃, that in which the group X is the formula (IV) and the group Y is —OCH₃, that in which the group X is the formula (V) and the group Y is —OCH₃, that in which the group X is the formula (VI) and the group Y is —CH₃, and that in which the group X is the formula (VII) and the group Y is —OCH₃, are commercially available from ALDRICH CO., and can be utilized.

The nitrogen-containing biphenyl derivative (I) described above is a quaternary ammonium salt derivative and is a nitrogen-containing polycyclic compound. The nitrogen-containing biphenyl derivative (I), even when added alone, is adsorbed to a high current portion such as the surface or convex portion of the substrate in a copper plating solution, and suppresses growing of plating in such portion. Thus, plating progresses better in a concave portion, that is, a low current portion. This can promote the growing of plating in the vias or through holes, which will enable filling.

The copper plating solution of the invention is prepared by adding the nitrogen-containing biphenyl derivative (I) described above in the basic composition copper plating solution. While the nitrogen-containing biphenyl derivative (I) can be added by combination with other substances to the basic composition copper plating solution, to expedite solution handling, etc., it is preferably added alone. Further, the concentration may be from 0.01 to 1,000 mg/L and, more preferably, from 20 to 100 mg/L.

The basic composition of the copper plating solution contains a copper ion ingredient and an anion ingredient, in which the copper ion ingredient is supplied from various copper-containing compounds. Examples of the copper-containing compound include copper sulfate, copper carbonate, copper oxide, copper chloride, inorganic acid salts of copper such as copper pyrophosphate, copper alkane sulfonates such as copper methane sulfonate and copper propane sulfonate, copper isethinate, copper alkanol sufonates such as copper propanol sulfonate, organic acid salts of copper such as copper acetate, copper citrate, and copper tartarate, as well as salts thereof. Among them, copper sulfate pentahydrate is relatively preferred in view of easy availability, cost, solubility, etc. One of these copper compounds can be used alone or a combination of two or more of them can be used. The concentration of the copper ions is from 100 to 300 g/L and, more preferably, from 200 to 250 g/L in a case of the copper sulfate pentahydrate.

Further, as the anion ingredient, acids capable of dissolving copper can be used in addition to counter ions of the copper-containing compounds. Preferred specific examples of such acid include sulfuric acid, alkane sulfonic acids such as methane sulfonic acid and propane sulfonic acid, alkanol sulfonic acid, and organic acids such as citric acid, tartaric acid, and formic acid. The organic acids or inorganic acids can be used each alone or in combination of two or more of them. The concentration of the organic acid or the inorganic acid is, preferably, from 10 to 200 g/L and, particularly, between 18 and 150 g/L in the copper plating solution composition.

Further, in the basic composition of the copper plating solution of the invention, halogen ions can be present as an electrolyte, and the presence of chlorine ions is particularly preferred. The chlorine ions are preferably from 10 to 100 mg/L and, more preferably, from 10 to 50 mg/L in terms of chlorine concentration. The chlorine ions serve to maintain the balance between the nitrogen-containing biphenyl derivative (I) which is a nitrogen-containing polycyclic compound, and the copper ions. That is, the chlorine ions have an effect of firmly adsorbing on a copper foil to improve the adsorption of the nitrogen-containing derivative (I) on a copper foil. It is often necessary that the chlorine ions are added generously in a case of using the nitrogen-containing biphenyl derivative (I) at a low concentration. However, in a case of use at a high concentration, since chlorine is contained in the additive itself, it is often unnecessary to add the chlorine ions.

pH of the basic composition of the copper plating solution is preferably acidic.

While the copper plating solution of the invention is prepared by adding the nitrogen-containing biphenyl derivative (I) to the basic composition copper plating solution, a sulfoalkyl sulfonic acid and salts thereof, bissulfo organic compound, or dithiocarbamic acid derivative can be incorporated also. They are additive components referred generally as brighteners, and specific examples thereof include the following:

(a) a sulfoalkyl sulfonic acid and a salt thereof represented by the following formula (XI):

HS—L₁—SO₃M₁ (XI)

(wherein L₁ represents a saturated or unsaturated alkylene group of 1 to 18 carbon atoms, and M₁ represents hydrogen or alkali metal),

(b) a bissulfo organic compound represented by the following formula (XII):

X₁—L₂—S—S—L₃—Y₁ (XII)

(wherein X₁ and Y₁ each represents a sulfate residue or phosphate residue, and L₂ and L₃ each represents a saturated or unsaturated alkylene group of 1 to 18 carbon atoms), and

(c) a dithiocarbamic acid derivative represented by the following formula (XIII):

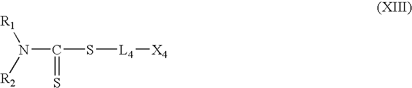

(wherein R₁ and R₂ each represents a hydrogen atom or a lower alkyl group of 1 to 3 carbon atoms, L₄ represents an alkylene group of 3 to 6 carbon atoms, and X₂ represents a sulfate residue or a phosphate residue).

One of the ingredients (a) to (c) above can be used alone, or a combination of two or more of them can be used. Further, the concentration thereof to be used is, preferably, from 0.1 to 200 mg/L and, more preferably, from 0.1 to 20 mg/L in the copper plating solution.

In the plating bath used in the invention, a hydrocarbon compound generally used in copper plating as shown by the formula (IX) can be conatined in addition to the ingredients described above.

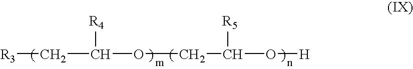

(wherein R₃ represents a higher alcohol residue of 8 to 25 carbon atoms, an alkylphenol residue having an alkyl group of 1 to 25 carbon atoms, an alkyl naphthol residue having an alkyl group of 1 to 25 carbon atoms, an aliphatic acid amide residue of 3 to 22 carbon atoms, an alkylamine residue of 2 to 4 carbon atoms, or a hydroxyl group, R₄ and R₅ each represents a hydrogen atom or a methyl group, and m and n each represents an integer of 1 to 100.

Specific examples of the hydrocarbon compound (IX) include 1,3-dioxolane polymer, polyethylene glycol, polypropylene glycol, pluronic type surfactant, polypropylenepropanol, polyethylene glycol derivatives such as polyethylene glycol/glyceryl ether and polyethylene glycol dialkyl ether, and oxyalkylene polymers.

Further, in the plating bath of the invention, a moistening agent with an aim of reducing the surface tension, copolymers of ethylene oxide and propylene oxide, etc. may also be incorporated.

Next, a method of manufacturing an electronic circuit substrate having fine copper wiring circuits using the plating solution of the invention described above (hereinafter referred to as “method of the invention”) is to be described.

In practicing the method of the invention, an electronic circuit substrate in which microholes or microgrooves in the shape of an electronic circuit wiring are formed at the surface (hereinafter referred to as “substrate”) is made electroconductive and the surface is cleaned and activated. As the substrate, a semiconductor wafer or PCB is used in which the microholes or microgrooves are at a micron or sub-micron order. Further, means for making the substrate electroconductive or for cleaning and activation of the substrate rendered electroconductive can be conducted by utilizing an already known method in accordance with the substrate to be used.

For practicing the method of the invention more specifically, for example, as shown in FIG. 2, a substrate 401 (for example, semiconductor wafer or PCB) having blind vias 403 and through holes 405 at a micron level or sub-micron level (both having hole diameter: 20 to 500 μm, aspect ratio: 1 to 5) is at first made electroconductive in accordance with an conventional method as a first step and then cleaned with 3% sulfuric acid and with pure water.

Then, the substrate 401 is dipped in a plating solution containing a copper ion ingredient, an anion ingredient, and a nitrogen-containing biphenyl derivative (I) as a single additive (hereinafter referred to as “additive”), and copper ions are deposited at a constant current density on the substrate 401 which is the cathode. The copper ions of the plating solution are supplied from copper-containing compounds such as copper sulfate, copper carbonate, copper oxide and copper sulfate pentahydrate.

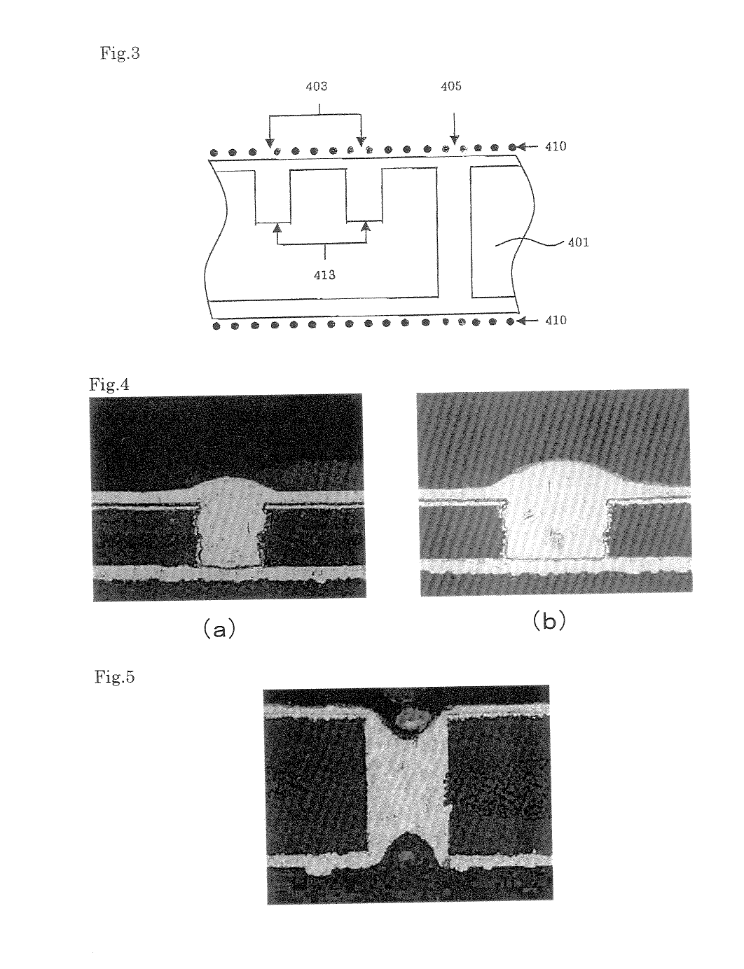

While the electroplating can be practiced in accordance with conditions set by existent copper plating, a favorable result is obtained by conducting preliminary current supply. That is, as shown in FIG. 3, when a preliminary current is passed, since the additive 410 is affected by the current distribution and is adsorbed more on the mirror surface 411 and corners at the hole top ends of the blind via holes 403 and through holes 405 in the substrate 401 and thus suppresses the diffusion speed of the additive 410, adsorption of the additive 410 to the bottom is suppressed. Accordingly, since the difference of concentration of the additive 410 is caused between the substrate surface and the bottom 413 of the blind vias 403 and the bottom 415 of the through hole 405, superfilling as shown in FIG. 4 can be attained due to the difference in the suppressing effect.

In the method of the invention, preferred conditions for filling the via holes and/or through holes with copper for plating using the nitrogen-containing biphenyl derivative (I) as a single additive are as shown below.

(1) For the additive used in the plating bath, only one of the nitrogen-biphenyl derivatives (I) is used.

(2) The composition of the plating bath comprises each of the following ingredients: CuSO₄·5H₂O, H₂SO₄, Cl⁻, and the additive with the ingredients given in (1) above.

(3) The concentration for each of the ingredients of the plating bath composition is as shown below.

(3-A) CuSO₄·5H₂O: 180 g/L to 250 g/L (standard concentration: 220 g/L, which should be changed depending on the diameter and the depth of the hole. For example, the copper concentration has to be increased as the diameter of the hole is larger or the depth of the hole is larger.

(3-B) H₂SO₄ (96%): from 20 to 80 g/L

(3-C) Cl⁻ (NaCl or HCl): 10-60 mg/L (standard: 20 mg/L, in a case where the chlorine concentration is 150 mg/L or more, it results in conformal deposition).

(3-D) Nitrogen-containing biphenyl derivative (I) compound: 0.01 to 100 mg/L

(3-E) Sulfur-containing compound (example: SPS): 0 to 100 ppm

(3-F) Polymeric hydrocarbon compound (example: polyethylene glycol (PEG)): 0 to 1,000 mg/L

(4) The plating bath temperature is about from 25 to 28° C.

(5) The current density is about from 0.16 to 1.97 A/dm².

The copper plating solution of the invention can be used for semiconductor or PCB plating having through holes or via holes at a micron or sub micron level and can fill them sufficiently.

Then, the filling according to the invention can be said to be superfilling which is much superior to the existent techniques. That is, in 2000, West presented, in the report entitled as Theory of Filling of High-Aspect Ratio Trenches and Vias in Presence of Additives, in the Journal of The Electrochemical Society, P 227-262, Vol. 147, No. 1, the simulation result that the proportion between the amount of single component additive consumed and the diffusion rate at the time of dissolution is constant and super-filling is possible in a case where a suppressor agent concentration at the upper edge of the hole is proportional to that at the bottom. There was no additive capable of satisfying these simulation requirements at that time, but the nitrogen-containing biphenyl derivative (I) (leveling agent) used in the present invention is an additive that can be used alone and since it has the effect of N⁺ functional group, it can be said to be an additive described in the simulation above. Accordingly, through holes and blind via holes can be filled by so-called superfilling.

### EXAMPLE

The present invention is to be described more specifically with reference to Examples. However, materials and, numerical values referred to in the Examples no way restrict the invention and the range of use can of course be changed in accordance with the purpose and the kind of the substrate.

### Example 1

### Filling Test for Blind via Hole (1):

Using as a test specimen an IC substrate having a blind via hole of 65 μm diameter and 60 μm depth (specimen 1) and an IC substrate having a blind via hole of 105 μm diameter and 60 μm depth (specimen 2) , a filling test for the blind via holes treated according to the plating method of the invention was conducted. The composition of the plating solution and the plating conditions are as shown below.

Copper Sulfate Plating Solution Composition:

| | | |
|---|---|---|
| Copper sulfate pentahydrate (CuSO₄•5H₂O): | 220 | g/L |
| Sulfuric acid (H₂SO₄): | 55 | g/L |
| Chlorine ion (Cl⁻) | 20 | mg/L |
| Additive: | | |
| Nitrogen-containing biphenyl derivative^{note 1)} | 40 | mg/L |

^{note 1)}In (I), X = formula (III), Y = —OCH₃

Plating Condition:

| | |
|---|---|
| Cathode current density: | 0.2425 A/dm² |
| Plating time: | 200 min |
| Plating solution temperature: | 25° C. |
| Stirring: | Not stirred |

Cross sectional observation images for the state of the specimen 1 and the specimen 2 after plating are shown in FIG. 4 (a) and FIG. 4(b) As apparent from these results, while voids and seams were often caused in the known plating baths each making use of a multi-component additive, resulting in the problem of poor filling efficiency, voids and seams were not observed after the plating according to the method of the invention, and satisfactory filling was obtained. This is considered to be due to the fact that as a result of using the nitrogen-containing biphenyl derivative (I) as a single additive, a concentration gradient formed between the inside of the hole and the surface of the hole due to the balance of charge absorption, consumption, and diffusion speed between electric fields, so as to attain an excellent filling effect.

### Example 2

### Filling Test for Through Hole:

Using an IC substrate having through hole of 85 μm diameter and 150 μm depth as a test specimen (specimen 3) a filling test for a through hole treated according to the plating method of the invention was conducted. The composition of the plating solution and the plating conditions are as shown below.

Copper Sulfate Plating Solution Composition:

| | | |
|---|---|---|
| Copper sulfate pentahydrate (CuSO₄•5H₂O): | 220 | g/L |
| Sulfuric acid (H₂SO₄): | 55 | g/L |
| Chlorine ion (Cl⁻) | 20 | mg/L |
| Additive: | | |
| Nitrogen-containing biphenyl derivative^{note 1)} | 40 | mg/L |

^{note 1)}Identical with those used in Example 1

Plating Condition:

| | |
|---|---|
| Cathode current density: | 0.2425 A/dm² |
| Plating time: | 200 min |
| Plating solution temperature: | 25° C. |
| Stirring: | Not stirred |

Cross sectional observation images of the specimen 3 in the state after plating are shown in FIG. 5. A satisfactory through hole filling performance was obtained where the concentration of the nitrogen-containing biphenyl derivative (I) as a single additive was from 20 to 100 ppm, and chlorine concentration was from 10 to 100 ppm.

### Example 3

### Filling Test for Blind via Hole (2)

Using the specimen 1 (IC substrate having a blind via hole of 65 μm diameter and 60 μm depth) and the specimen 2 (IC substrate having a blind via hole of 105 μm diameter and 60 μm depth) of Example 1, filling tests for the blind via hole were conducted with different plating solutions. The composition of the plating solutions and the plating conditions are as shown below.

Copper Sulfate Plating Solution Composition:

| | | |
|---|---|---|
| Copper sulfate pentahydrate (CuSO₄•5H₂O): | 220 | g/L |
| Sulfuric acid (H₂SO₄): | 55 | g/L |
| Chlorine ion (Cl⁻) | 60 | mg/L |
| Additive: | | |
| Nitrogen-containing biphenyl derivative (I)^{note 2)} | 40 | mg/L |
| SPS^{note 3)} | 0.3 | mg/L |

^{note 2)}in the formula (I), X = formula (II), Y = —H
^{note 3)}in the formula (XI), L₂ = L₃ = —C₃H₆—, X₁ = Y₁ = —SO₃

Plating Condition:

| | |
|---|---|
| Cathode current density and | 0.97 A/dm², |
| plating time: | 30 min → 1.94 A/dm², 55 min |
| Plating solution temperature: | 25° C. |
| Stirring: | Not stirred |

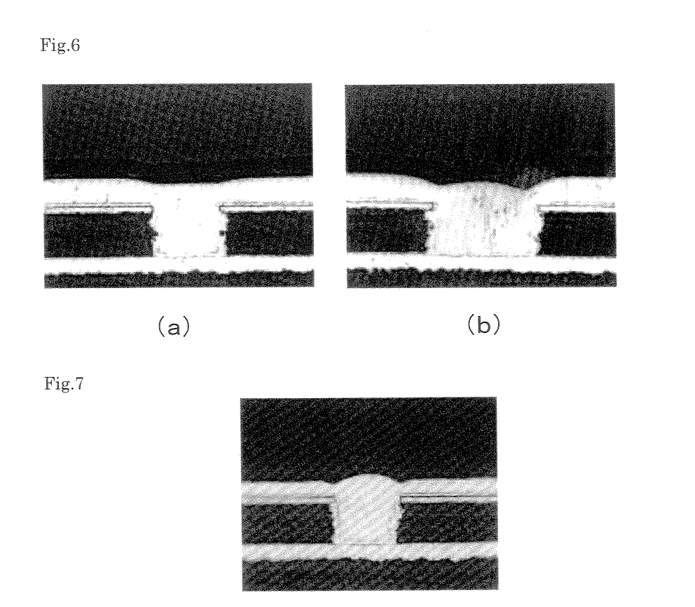

Cross sectional observation images for the state of the specimen 1 and specimen 2 after plating in Example 3 are shown in FIG. 6(a) and FIG. 6(b). In this example, the current density could be increased from 0.2425 A/dm² to 0.97-1.94 A/dm². Usually, nodules are generated on the surface of the substrate at such a high current density but by the addition of SPS as the brightener, nodules were not generated and a satisfactory result was obtained.

### Example 4

### Filling Test for Blind via Hole (3)

Using the specimen 1 (IC substrate having a blind via hole of 65 μm diameter and 60 μm depth) of Example 1, a filling test for the blind via hole was conducted while changing the plating solution. The composition of the plating solution and the plating conditions are as shown below.

Copper Sulfate Plating Solution Composition:

| | | |
|---|---|---|
| Copper sulfate pentahydrate (CuSO₄•5H₂O): | 220 | g/L |
| Sulfuric acid (H₂SO₄): | 55 | g/L |
| Chlorine ion (Cl⁻) | 60 | mg/L |
| Additive: | | |
| Nitrogen-containing biphenyl derivative (I)^{note 4)} | 1 | mg/L |
| SPS^{note 3)} | 1 | mg/L |
| PEG^{note 5)} | 200 | mg/L |

^{note 4)}in the formula (I), X = formula (II), Y = —H
^{note 5)}in the formula (XI), L₂ = L₃ = —C₃H₆—, X₁ = Y₁ = —SO₃
^{note 6)}polyethylene glycol (average molecular weight: 8000)

Plating Condition

| | |
|---|---|
| Cathode current density and | 0.97 A/dm², |
| plating time: | 15 min → 1.94 A/dm², 30 min |
| Plating solution temperature: | 25° C. |
| Stirring: | Not stirred |

Cross sectional observation images for the state of the specimen 1 after plating in Example 4 are shown in FIG. 7. In this Example, it was shown that a favorable filling performance was obtained even when a polymer ingredient (PEG) was added in addition to the nitrogen-containing biphenyl derivative (I).

### Example 5

### Copper Plating Solution for Filling (1):

To a basic composition copper sulfate plating solution comprising 220 g/L of copper sulfate pentahydrate, 55 g/L of sulfuric acid, and 60 mg/L of chlorine ions, 50 mg/L of a nitrogen-containing biphenyl derivative (group X=(III), group Y=—OCH₃) was added as an additive to form a copper plating solution for filling.

### Example 6

### Copper Plating Solution for Filling (2):

To a basic composition copper sulfate plating solution comprising 220 g/L of copper sulfate pentahydrate, 55 g/L of sulfuric acid, and 60 mg/L of chlorine ions, 50 mg/L of a nitrogen-containing biphenyl derivative (group X=(IV), group Y=—OCH₃), 1 mg/L of SPS, and 400 mg/L of PEG were added as additives to form a copper plating solution for filling.

### Example 7

### Copper Plating Solution for Filling (3):

To a basic composition copper sulfate plating solution comprising 225 g/L of copper sulfate pentahydrate, 55 g/L of sulfuric acid, and 60 mg/L of chlorine ions, 40 mg/L of a nitrogen-containing biphenyl derivative (group X=(V), group Y=—OCH₃) was added as an additive to form a copper plating solution for filling.

### Example 8

### Copper Plating Solution for Filling (4):

To a basic composition copper sulfate plating solution comprising 225 g/L of copper sulfate pentahydrate, 55 g/L of sulfuric acid, and 60 mg/L of chlorine ions, 60 mg/L of a nitrogen-containing biphenyl derivative (group X=(V), group Y=—OCH₃) and 15 mg/L of SPS were added as an additive to form a copper plating solution for filling.

### Example 9

### Copper Plating Solution for Filling (5):

To a basic composition copper sulfate plating solution comprising 220 g/L of copper sulfate pentahydrate, 55 g/L of sulfuric acid, and 60 mg/L of chlorine ions, 50 mg/L of a nitrogen-containing biphenyl derivative (group X=(VI), group Y=—CH₃) , was added as an additive to form a copper plating solution for filling.

### Example 10

### Copper Plating Solution for Filling (6):

To a basic composition copper sulfate plating solution comprising 220 g/L of copper sulfate pentahydrate, 55 g/L of sulfuric acid, and 60 mg/L of chlorine ions, 40 mg/L of a nitrogen-containing biphenyl derivative (group X=(VII), group Y=—OCH₃) and 1 mg/L of SPS were added as an additive to form a copper plating solution for filling.

### INDUSTRIAL APPLICABILITY

The nitrogen-biphenyl derivative (I) as the effective ingredient of the additive for copper plating according to the invention can fill microholes or microgroove even when it is the only component added to the basic composition copper plating solution, and control of the additive can be carried out more easily compared with conventional copper plating using multiple additives.

Further, the copper plating solution containing the nitrogen-containing biphenyl derivative (I) enables void-free filling of both through holes and blind via holes at the micron level or sub-micron level, and can be utilized effectively in the manufacture of electronic circuit substrates having fine copper wiring circuits.

### BRIEF DESCRIPTION OF THE DRAWINGS

[FIG. 1] FIG. 1 is a view schematically showing a cross section of filled metal wirings by an existent technique.

[FIG. 2] FIG. 2 is a view schematically showing the state of a substrate before treatment with the method of the invention.

[FIG. 3] FIG. 3 is a view schematically showing the state of a substrate after treatment with the method of the invention.

[FIG. 4] FIG. 4 is a view showing cross sectional observation images (200×) of blind via holes of an IC substrate after plating in Example 1. In the drawing, (a) is specimen 1, and (b) is specimen 2.

[FIG. 5] FIG. 5 is a view showing cross sectional observation images (200×) of through holes of an IC substrate after plating according to Example 2.

[FIG. 6] FIG. 6 is a view showing cross sectional observation images (200×) of blind via holes of an IC substrate after plating according to Example 3. In the drawings, (a) is specimen 1, and (b) is specimen 2.

[FIG. 7] FIG. 7 is a view showing cross sectional observation images (200×) of the blind via holes of the IC substrate of specimen 1 after plating in Example 4.

### DESCRIPTION OF THE REFERENCE NUMERALS AND SIGNS

101: material
103: blind via
105: metal layer
111: void
113: seam
115: super filling
401: substrate
403: blind via
405: through hole
410: additive
411: surface
413: bottom

1. An additive for copper plating comprising, as an effective ingredient, a nitrogen-containing biphenyl derivative represented by the following formula (I): 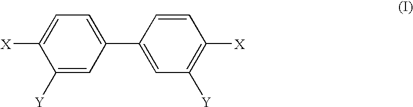
[wherein X represents a group selected from the following groups (II)-(VII): 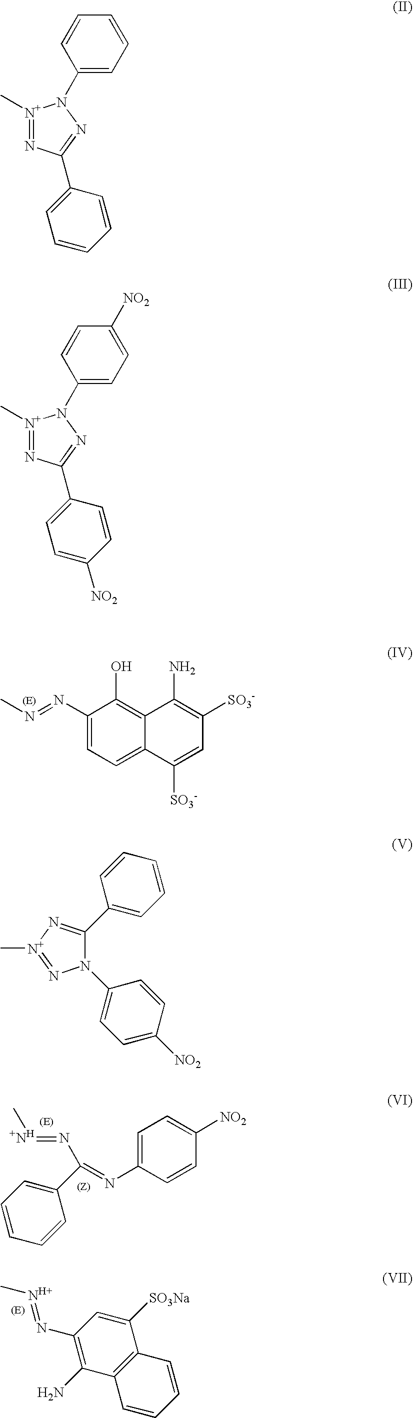
and Y represents a lower alkyl group, lower alkoxy group, nitro group, amino group, sulfonyl group, cyano group, carbonyl group, 1-pyridyl group, or the formula (VIII): 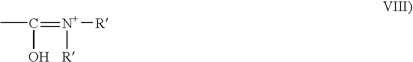
(wherein R′ represents a lower alkyl group)].

2. An additive for copper plating according to claim 1 for use in filling microholes or microgrooves.

3. An additive for copper plating according to claim 1 or 2, wherein a nitrogen-containing biphenyl derivative is added such that its concentration in a basic composition copper plating solution is from 0.01 to 1,000 mg/L.

4. An additive for copper plating according to claim 1 or 2, wherein a nitrogen-containing biphenyl derivative is added such that its concentration in a basic composition copper plating solution is from 20 to 100 mg/L.

5. A copper plating solution formed by adding an additive for copper plating comprising, as an effective ingredient, a nitrogen-containing biphenyl derivative represented by the following formula (I): 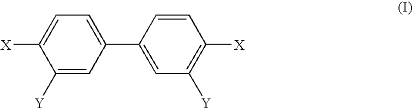
[wherein X represents a group selected from the following groups (II)-(VII): 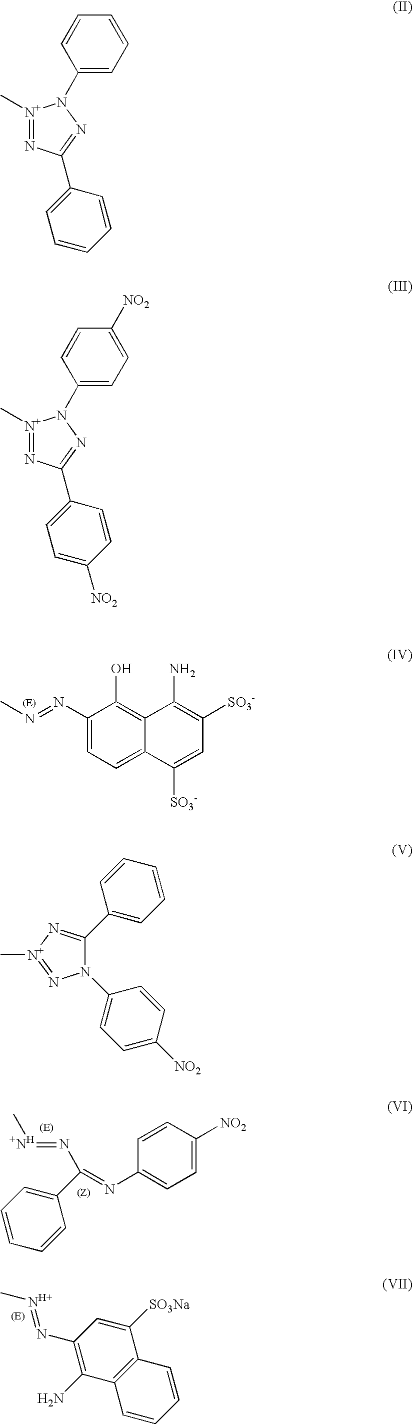
and Y represents a lower alkyl group, lower alkoxy group, a nitro group, amino group, sulfonyl group, cyano group, carbonyl group, cyano group, carbonyl group, 1-pyridyl group, or the formula (VIII): 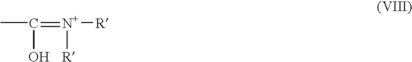
(wherein R′ represents a lower alkyl group) to a basic composition copper plating solution containing a copper ion ingredient and an anion ingredient.

6. A copper plating solution according to claim 5, for filling microholes or microgrooves.

7. A copper plating solution according to claim 5 or 6, wherein the addition amount of the nitrogen-containing biphenyl derivative is such that its concentration in the basic composition copper plating solution is from 0.01 to 1,000 mg/L.

8. A copper plating solution according to claim 5 or 6, wherein the addition amount of the nitrogen-containing biphenyl derivative is such that its concentration in the basic composition copper plating solution is from 20 to 100 mg/L.

9. A copper plating solution according to any one of claims 5 to 8, wherein copper sulfate, copper carbonate, copper oxide, copper chloride, copper pyrophosphate, copper alkane sulfonate, copper alkanol sulfonate, copper acetate, copper citrate, copper tartarate is used as a copper ion source.

10. A copper plating solution according to any one of claims 5 to 8, wherein copper sulfate is used as a copper ion source.

11. A copper plating solution according to claim 10, wherein copper sulfate pentahydrate as the copper ion source is used within a range from 100 to 300 g/L (25 to 75 g/L copper ion concentration) in the basic composition copper plating solution.

12. A copper plating solution according to claim 10, wherein copper sulfate pentahydrate as a copper ion source is used within a range from 200 to 250 g/L (50 to 62.5 g/L copper ion concentration) in the basic composition copper plating solution.

13. A copper plating solution according to any one of claims 5 to 12, wherein a halogen ion is further contained as an electrolyte.

14. A copper plating solution according to claim 13, wherein the halogen ion is a chlorine ion and the concentration thereof in the basic composition copper plating solution is from 10 to 100 mg/L.

15. A copper plating solution according to any one of claims 5 to 14, wherein at least one acid is contained as an anionic ingredient source.

16. A copper plating solution according to claim 15, wherein the acid is sulfuric acid and the concentration thereof in the basic composition copper plating solution is from 18 g/L −150 g/L.

17. A copper plating solution according to claims 5 to 16, wherein at least one sulfur-containing compound is further contained.

18. A copper plating solution according to claim 17, wherein one or more sulfur-containing compounds are selected from the group consisting of sulfoalkyl sulfonic acids and salts thereof, bissulfo organic compounds, and dithiocarbamic acid derivatives, and the concentration of the compound is from 0.1 to 200 mg/L.

19. A copper plating solution according to any one of claims 5 to 18, wherein at least one polymeric hydrocarbon compound is further contained.

20. A copper plating solution according to claim 19, wherein the concentration of the polymeric hydrocarbon compound in the basic composition copper plating solution is from 10 to 2,000 mg/L.

21. A copper plating solution according to claim 19 or 20, wherein the polymeric hydrocarbon compound is a compound represented by the following formula (IX): 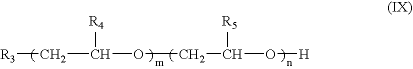
(wherein R₃ represents a higher alcohol residue of 8 to 25 carbon atoms, an alkylphenol residue having an alkyl group of 1 to 25 carbon atoms, an alkyl naphtol residue having an alkyl group of 1 to 25 carbon atoms, an aliphatic acid amide residue of 3 to 22 carbon atoms, an alkylamine residue of 2 to 4 carbon atoms, or a hydroxyl group, and R₄ and R₅ each represents a hydrogen atom or a methyl group, and m and n each represents an integer of 1 to 100).

22. A copper plating solution according to claim 19 or 20, wherein one or more polymeric hydrocarbon compounds are selected from the group consisting of 1,3-dioxolane polymer, polyethylene glycol, polypropylene glycol, pluronic type surfactant, polypropylenepropanol, polyethylene glycol derivatives such as polypethylene glycol/glyceryl ether and polyethylene glycol/dialkyl ether, and oxyalkylene polymers.

23. A method of manufacturing an electronic circuit substrate having a fine copper wiring circuit, which comprises electroplating using an electronic circuit substrate in which microholes or microgrooves in the shape of electronic circuit wirings are formed on the surface as a cathode in a copper plating solution containing a copper ion ingredient, an anion ingredient, and a nitrogen-containing biphenyl derivative represented by the following formula (I): 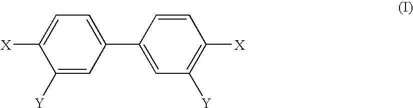
[wherein X represents a group selected from the followings groups (II)-(VII), 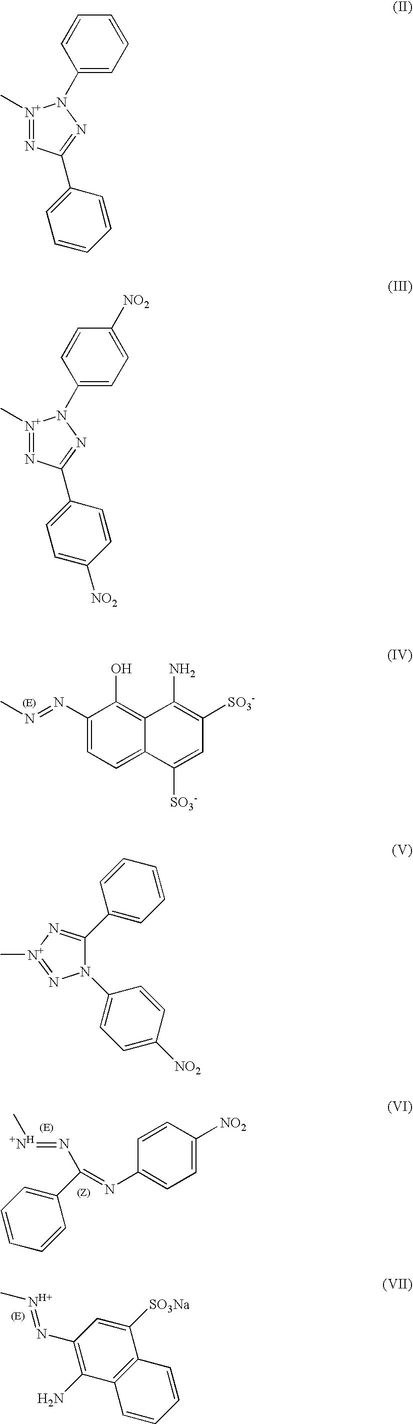
and Y represents a lower alkyl group, lower alkoxy group, nitro group, amino group, sulfonyl group, cyano group, carbonyl group, 1-pyridyl group, or the formula (VIII): 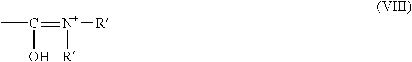
(wherein R′ represents a lower alkyl group)].
